(19) United States
(12) Patent Application Publication
(10) Pub. No.: US 2023/0406825 A1
(43) Pub. Date: Dec. 21, 2023

(54) COMPOUNDS, COMPOSITIONS THEREOF, AND METHODS FOR TREATING ER+ BREAST CANCER

(71) Applicant: The Board of Regents of The University of Texas System, Austin, TX (US)

(72) Inventors: Murat Can Cobanoglu, Dallas, TX (US); Ganesh Raj, Dallas, TX (US)

(73) Assignee: The Board of Regents of The University of Texas System, Austin, TX (US)

(21) Appl. No.: 18/251,152

(22) PCT Filed: Nov. 2, 2021
(86) PCT No.: PCT/US2021/057684

§ 371 Date: Apr. 28, 2023

Related U.S. Application Data
(60) Provisional application No. 63/108,750, filed on Nov. 2, 2020.

(51) Int. Cl. C07D 211/94; A61P 35/00

(52) U.S. Cl. CPC C07D 211/94; A61P 35/00

(57) ABSTRACT

Compounds and methods are disclosed for the treatment and/or amelioration of breast cancer. The compounds and methods may treat and/or ameliorate breast cancer by way of activity on isocitrate dehydrogenase 1 (IDH1). The breast cancer may be estrogen receptor positive (ER+) breast cancer. Also disclosed are compositions, including pharmaceutical compositions, for the treatment and/or amelioration of breast cancer.

### CROSS-REFERENCE TO RELATED APPLICATION

This application claims the benefit of U.S. Provisional Patent Application Ser. No. 63/108,750 filed on Nov. 2, 2020, the content of which is incorporated by reference in its entirety

### BACKGROUND

### 1. Field

The present disclosure generally relates compounds and compositions thereof for the treatment of ER+ breast cancer. Aspects of the disclosure also relate to methods of treating ER+ breast cancer.

### 2. Discussion of Related Art

Breast cancer will be diagnosed in about 13% of women in the U.S. in their lifetimes, and more than 3% will die from the disease. Worldwide, breast cancer is now the leading cause of cancer mortality in women, accounting for more than 620,000 deaths per year. In 2018, more than 2 million new cases were diagnosed worldwide, with more than 270,000 of these in the U.S. alone.

Cancerous tumors can arise in any tissue of the breast, but most commonly in epithelial tissue of the lobules and ducts. The epithelial cells are separated from the connective tissue surrounding the lobules and ducts by a layer of extracellular material known as the basement membrane. Tumors that are limited by the basement membrane, but may proliferate in the lumen of a lobule or duct, are referred to as lobular carcinoma in situ (LCIS) or ductal carcinoma in situ (DCIS). LCIS is typically not detected by examination or mammography, whereas DCIS tumors often develop central necrosis and calcify, becoming clinically palpable and detectable by mammography. DCIS is more likely than LCIS to be malignant and to become invasive.

Breast tumors often, but do not always, have hormone receptors, more particularly estrogen and progesterone receptors that can be detected in tissue samples obtained by biopsy. A tumor in which the expression of estrogen receptors (ER) is identified is said to be estrogen receptor positive (ER+), and one lacking ER expression is said to be estrogen receptor negative (ER−). Likewise, tumors can be progesterone receptor positive (PR+) or negative (PR−), based on the detectable level of PR expression. Tumors that are ER+ and/or PR+ typically show an increase in rate of proliferation in presence of these respective hormones, which occur naturally in the female body and may be supplemented artificially, for example in hormone replacement therapy (HRT). About 70% of all primary human breast cancers are ER+ and the great majority of these are also PR+.

ER+ breast cancer is often treatable with drugs that bind more or less selectively to ER. Such drugs partially or completely prevent estrogen from binding to ER or directly antagonize the ER and thereby modulating a cascade of events blocking cell proliferation and tumor growth. Tamoxifen was the first, and is still most widely used, of a class of such drugs known as selective estrogen receptor modulators (SERMs). SERMs are useful not only in palliative treatment of ER+ breast cancer but have marked prophylactic utility in healthy subjects at high risk of developing breast cancer, for example subjects having family history of the disease or a previous finding of atypical hyperplasia or in situ carcinoma in a breast tissue biopsy. Other risk factors include advanced age (e.g., 60 years or older), nulliparity and early menarche. For instance, tamoxifen is widely prescribed for women having one or more risk factors and has been found in extensive studies to reduce incidence of invasive breast cancer, for example by almost 50% when administered for 5 years in the Breast Cancer Prevention Trial (P-1) initiated in 1992. See Fisher et al. (1998) J. Natl Cancer Inst. 90(18):1371-1388.

Unfortunately, SERMs are not universally effective in durably treating breast cancer. Aside from lacking any activity in ER− cancers, it is now well established that even ER+ cancers can become resistant to SERM therapy. About 40% of ER+ breast cancer patients do not respond to anti-hormone therapy. See for example Biswas et al. (1998) Mol. Med. 4(7):454-467.

Another option for the treatment of ER+ invasive breast cancer is that SERM-resistant may be the estrogen receptor antagonist fulvestrant (ICI 182,780), which is believed to down-regulate ER expression in ER+ tumors. See, e.g., Robertson et al. (2001) Cancer Res. 61:6739-6746.

Yet another approach to treatment of estrogen-sensitive breast cancer is to reduce the level of estrogen circulating in the patient and thereby reduce the amount of estrogen available for binding to ER in breast tissue. This can be accomplished, e.g., by inhibition of aromatase, an enzyme involved in biosynthesis of estrogen from androgens. Aromatase inhibitors such as anastrozole, exemestane and letrozole are available for treatment of ER+ invasive breast cancer including such cancer that is or has acquired resistance to SERM therapy.

A body of literature now implicates the peptide angiotensin II (Ang II), a major regulator of blood pressure and cardiovascular homeostasis, in regulation of cell proliferation, angiogenesis, inflammation and tissue remodeling, and it has been suggested that Ang II might also play a role in cancer. See, e.g., Deshayes & Nahmias (2005) Trends Endocrinol. Metab. 16(7):293-299. Ang II exerts its bioregulatory effects through interaction with two major types of receptor located on the surface of target cells. These receptors, referred to as Ang II type 1 and type 2 (respectively AT1 and AT2) receptors, have been shown to be expressed in a variety of tissues.

A number of AT1 receptor antagonists and prodrugs thereof, including candesartan, eprosartan, irbesartan, losartan, ohnesartan, telmisartan and valsartan, have been developed for treatment of hypertension, and other useful properties have been identified for these agents. A list of AT1 receptor antagonists and prodrugs thereof may be found in U.S. Pat. No. 6,174,910, which is incorporated herein in its entirety for all purposes.

### BRIEF SUMMARY

The medical and pharmaceutical industries have focused on treating ER+ breast cancer using the pathways and activities discussed above. The inventors surprisingly discovered that certain compounds may be therapeutically beneficial for treating and/or ameliorating ER+ breast cancer by way of activity on IDH1.

According to a first aspect of the disclosure, provided are compounds of formula (I), acids thereof, and/or salts thereof:

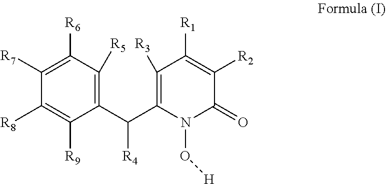

wherein
   R₁ is a methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl, and
   R₂, R₃, R₄, R₅, R₆, R₇, R₈, and R₉, are independent selected from hydrogen, methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl.

Preferably, R₁ is a methyl. Additionally or alternatively, R₂, R₃, R₄, R₅, R₆, R₇, R₈, and R₉, may be independently selected from hydrogen or methyl. In some cases, at least one of R₂, R₃, R₄, R₅, R₆, R₇, R₈, and R₉ is hydrogen. Preferably, wherein R₂, R₃, R₄, R₅, R₆, R₇, R₈, and R₉ are each hydrogen.

In at least one embodiment, the compound of Formula (I), a salt thereof, and/or an acid thereof has a structure according to Formula (II):

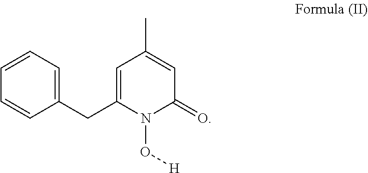

In accordance with another aspect of the disclosure, provided are compositions comprising an amount a compound of formula (I), acids thereof, and/or salts thereof:

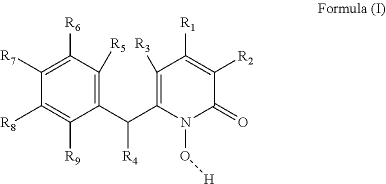

wherein
   R₁ is a methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl, and
   R₂, R₃, R₄, R₅, R₆, R₇, R₈, and R₉, are independent selected from hydrogen, methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl.

The composition may further comprise at least one excipient. Preferably, the amount of the compound of formula (I), an acid thereof, and/or a salt thereof present in the composition is a therapeutically effective amount.

According to yet a further aspect of the disclosure, provided are methods of treating and/or ameliorating breast cancer in a subject in need thereof comprising: administering a therapeutically effect amount of a compound of formula (I), an acid thereof, or a salt thereof, wherein the compound of formula (I) has the structure:

wherein

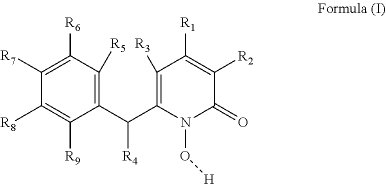

R₁ is a methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl, and
R₂, R₃, R₄, R₅, R₆, R₇, R₈, and R₉, are independent selected from hydrogen, methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl.

Preferably, R₁ is a methyl. Additionally or alternatively, R₂, R₃, R₄, R₅, R₆, R₇, R₈, and R₉, may be independently selected from hydrogen or methyl. In some cases, at least one of R₂, R₃, R₄, R₅, R₆, R₇, R₈, and R₉ is hydrogen. Preferably, wherein R₂, R₃, R₄, R₅, R₆, R₇, R₈, and R₉ are each hydrogen.

In some embodiments, the method involves treating and/or ameliorating ER+ breast cancer.

In some embodiments, the compound of Formula (I) has a structure according to Formula (II):

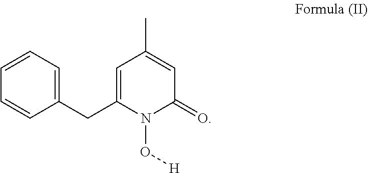

### BRIEF DESCRIPTION OF DRAWINGS

Implementation of the present technology will now be described, by way of example only, with reference to the attached figures, wherein:

FIG. 1 is a graph showing the effect of the compounds of Formula (II) on MCF-7, ZR-75 and T-47D breast cancer cells in accordance with aspects of the disclosure.

FIG. 2 is a graph showing the effect of the compounds of Formula (II) on MCF-7 (a triple negative breast cancer lines) and MDA-MB-231 breast cancer cell lines according to aspects of the disclosure.

FIG. 3 is a graph showing the effect of the compounds of Formula (II) in comparison to vorasidenib and ivosidenib on breast cancer cells in accordance to aspects of the disclosure.

FIG. 4 is a schematic of the steps for growing cancerous breast tissues on a gelatin sponge according to aspects of the disclosure.

FIG. 5 is images of ER-positive breast tumors samples after administration of the compounds of Formula (II) and dimethylsulfoxide (“DMSO”) in accordance with aspects of the disclosure.

FIG. 6 are graphs showing the response of ER-positive breast tumors samples to administration of compounds of Formula (II) and DMSO according to aspects of the disclosure.

FIG. 7 is a graph showing the proliferation or retardation of breast cancer cells to administration of compounds of Formula (II) and DMSO in accordance with aspects of the disclosure.

FIGS. 8 and 9 are graphs showing the effect of BMS-309403 or Z1445513748 compounds on MCF-7, ZR-75, and T-47D breast cancer cells in accordance with aspects of the disclosure.

It should be understood that the various aspects are not limited to the arrangements and instrumentality shown in the drawings.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Thus, references to one or an embodiment in the present disclosure can be references to the same embodiment or any embodiment; and, such references mean at least one of the embodiments.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

As used herein, the terms “comprising,” “having,” and “including” are used in their open, non-limiting sense. The terms “a,” “an,” and “the” are understood to encompass the plural as well as the singular. Thus, the term “a mixture thereof” also relates to “mixtures thereof.”

Generally, the ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc. Other than in the operating examples, or where otherwise indicated, all numbers expressing quantities of ingredients and/or reaction conditions may be modified in all instances by the term “about,” meaning within +/−5% of the indicated number.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Aspects of the present disclosure generally relate to compounds and compositions thereof for the treatment of ER+ breast cancer. As noted above, the inventors unexpectedly discovered that certain compounds may be therapeutically beneficial for treating and/or ameliorating ER+ breast cancer due to activity on IDH1.

According to a first aspect of the disclosure, provided are compounds of formula (I), acids thereof, salts thereof, or a mixture thereof:

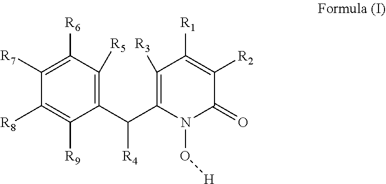

wherein
   R₁ is a methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl, and
   R₂, R₃, R₄, R₅, R₆, R₇, R₈, and R₉, are independent selected from hydrogen, methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl.

The compounds of Formula (I) may have a structure where R₇ is a C₂ to C₁₀ unsubstituted or substituted aryl, such as C₂ to C₈ aryl, C₃ to C₈ aryl, C₄ to C₈ aryl, C₅ to C₈ aryl, C₆ to C₈ aryl, C₇ to C₈ aryl, C₅ to C₇ aryl, or C₅ to C₆ aryl. R₁ of the compounds of Formula (I) may be C₁ to C₁₀ unsubstituted or substituted alkyls including, e.g., C₁ to C₉ alkyls, C₁ to C₈ alkyls, C₁ to C₇ alkyls, C₁ to C₆ alkyls, C₁ to C₅ alkyls, C₁ to C₄ alkyls, C₁ to C₃ alkyls, C₁ to C₂ alkyls, C₂ to C₆ alkyls, C₃ to C₆ alkyls, C₄ to C₅ alkyls, or C₅ to C₆ alkyls. In some instances, R₁ may be methyl, ethyl, n-propyl, i-propyl, n-butyl, or t-butyl. R₁ of the compounds of Formula (I) may be C₃ to C₁₀ unsubstituted or substituted cycloalkyl, such as C₃ to C₉ cycloalkyl, C₃ to C₈ cycloalkyl, C₃ to C₇ cycloalkyl, C₃ to C₆ cycloalkyl, C₃ to C₅ cycloalkyl, C₃ to C₄ cycloalkyl, C₄ to C₁₀ cycloalkyl, C₅ to C₁₀ cycloalkyl, C₆ to C₁₀ cycloalkyl, C₇ to C₁₀ cycloalkyl, C₈ to C₁₀ cycloalkyl, or C₉ to C₁₀ cycloalkyl. In at least one embodiment, R₁ is a methyl.

Additionally or alternatively, R₂, R₃, R₄, R₅, R₆, R₇, R₈, and R₉, may be C₂ to C₁₀ unsubstituted or substituted aryl, such as C₂ to C₈ aryl, C₃ to C₈ aryl, C₄ to C₈ aryl, C₅ to C₈ aryl, C₆ to C₈ aryl, C₇ to C₈ aryl, C₅ to C₇ aryl, or C₅ to C₆ aryl. The compounds of Formula (I) may have a structure where R₂, R₃, R₄, R₅, R₆, R₇, R₈, and R₉, are independently selected from C₁ to C₁₀ unsubstituted or substituted alkyls including, e.g., C₁ to C₉ alkyls, C₁ to C₈ alkyls, C₁ to C₇ alkyls, C₁ to C₆ alkyls, C₁ to C₅ alkyls, C₁ to C₄ alkyls, C₁ to C₃ alkyls, C₁ to C₂ alkyls, C₂ to C₆ alkyls, C₃ to C₆ alkyls, C₄ to C₅ alkyls, or C₅ to C₆ alkyls. In some instances, one or more of R₂, R₃, R₄, R₅, R₆, R₇, R₈, and R₉ are independently methyl, ethyl, n-propyl, i-propyl, n-butyl, or t-butyl. R₂, R₃, R₄, R₅, R₆, R₇, R₈, and R₉ of the compounds of Formula (I) may be C₃ to C₁₀ unsubstituted or substituted cycloalkyl, such as C₃ to C₉ cycloalkyl, C₃ to C₈ cycloalkyl, C₃ to C₇ cycloalkyl, C₃ to C₆ cycloalkyl, C₃ to C₅ cycloalkyl, C₃ to C₄ cycloalkyl, C₄ to C₁₀ cycloalkyl, C₅ to C₁₀ cycloalkyl, C₆ to C₁₀ cycloalkyl, C₇ to C₁₀ cycloalkyl, C₈ to C₁₀ cycloalkyl, or C₉ to C₁₀ cycloalkyl.

In some cases, R₂, R₃, R₄, R₅, R₆, R₇, R₈, and R₉, are independently selected from hydrogen or methyl. In some cases, at least one of R₂, R₃, R₄, R₅, R₆, R₇, R₈, and R₉ is hydrogen. Preferably, wherein R₂, R₃, R₄, R₅, R₆, R₇, R₈, and R₉ are each hydrogen.

In at least one embodiment, the compound of Formula (I), a salt thereof, and/or an acid thereof has a structure according to Formula (II):

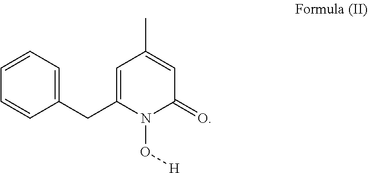

In accordance with another aspect of the disclosure, provided are compositions comprising an amount a compound of formula (I), acids thereof, and/or salts thereof:

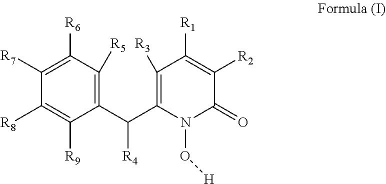

wherein
   R₁ is a methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl, and
   R₂, R₃, R₄, R₅, R₆, R₇, R₈, and R₉, are independent selected from hydrogen, methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl.

Preferably, the amount of the compound of formula (I), an acid thereof, and/or a salt thereof present in the composition is therapeutically effective. As used herein “therapeutically effective amount” or “therapeutically effective dosage” refers to an amount that is effective to achieve a desired therapeutic result. In some embodiments, the desired therapeutic result is the retarded and/or prevented growth of breast cancer cells, and particularly ER+ breast cancer cells. In other embodiments, the desired therapeutic result is a reduction of breast cancer cells (e.g., ER+ breast cancer cells). For example, the therapeutically effective amount may be an amount that reduces breast cancer cells (e.g., ER+ breast cancer cells) by at least 10%, preferably 20% or more, preferably 25% or more, preferably 30% or more, preferably 35% or more, preferably 40% or more, preferably 45% or more, preferably 50% or more, etc.

In some cases, the amount of compound of Formula (I) present in composition is more than about 1 μg. For example, the composition may comprise an amount of compounds of Formula (I) of about 2 μg or more, about 5 μg or more, about 10 μg or more, about 100 μg or more, about 500 μg or more, about 1000 μg or more, about 1500 μg or more, about 2000 μg or more, about 2500 μg or more, about 3000 μg or more, about 3500 μg or more, about 4000 μg or more, about 4500 μg or more, about 5000 μg or more, about 5500 μg or more, about 6000 μg or more, about 6500 μg or more, about 7000 μg or more, about 7500 μg or more, about 8000 μg or more, about 8500 μg or more, about 9000 μg or more, about 9500 μg or more, about 10 mg or more, about 20 mg or more, about 30 mg or more, about 40 mg or more, about 50 mg or more, about 60 mg or more, about 70 mg or more, about 80 mg or more, about 90 mg or more, about 100 mg or more, about 150 mg or more, about 200 mg or more, about 250 mg or more, about 300 mg or more, about 350 mg or more, about 400 mg or more, about 450 mg or more, about 500 mg or more, about 550 mg or more, about 600 mg or more, about 650 mg or more, about 700 mg or more, about 800 mg or more, about 900 mg or more, or about 1 g or more.

The composition may further comprise at least one excipient. Suitable excipients include pharmaceutically acceptable excipients, such as diluents, binders, fillers, buffering agents, pH modifying agents, disintegrants, dispersants, preservatives, lubricants, taste-masking agents, flavoring agents, coloring agents, or combinations thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

The compositions disclosed herein may be formulated into various dosage forms and administered by a number of different means that will deliver a therapeutically effective amount of the active ingredient. The excipients included in the compositions comprising compounds of Formula (I), acids thereof, and/or salts thereof may be based on the form of administering such compositions. Such compositions may be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term, “parenteral,” as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980).

Additionally, aspects of the disclosure relate to methods for treating breast cancer. Typically, the methods include administering an amount of the compositions comprising a compound of Formula (I), acids thereof and/or salts thereof. The amount of the compositions disclosed herein that is administered to the individual can and will vary depending upon the type or form of such compositions, the individual, the degree of cancer, and the particular mode of administration. Those skilled in the art will appreciate that dosages may also be determined with guidance from Goodman & Goldman's The Pharmacological Basis of Therapeutics, Tenth Edition (2001), Appendix II, pp. 475-493, and the Physicians' Desk Reference.

For oral administration, the method may include administering an amount of the composition disclosed herein in the form of a solid dosage or a liquid dosage. Solid dosage forms for oral administration include capsules, tablets, caplets, pills, powders, pellets, and granules. In such solid dosage forms, the compounds of Formula (I) are, typically, combined with one or more excipients, such as those described above. Liquid dosages of the composition disclosed herein may be in the form of aqueous suspensions, elixirs, or syrups.

For parenteral administration, the dosage of the compositions disclosed herein may be an aqueous solution, an oil-based solution, or in the form of a solid dosage. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as ethylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. In some instances, parental administration may be subcutaneous, intravenous, intramuscular, or intrasternal injection, or infusion.

The compounds described herein may have asymmetric centers. Compounds of the present disclosure containing an asymmetrically substituted atom may be isolated in optically active or racemic form. All chiral, diastereomeric, racemic forms and all geometric isomeric forms of a structure are intended, unless the specific stereochemistry or isomeric form is specifically indicated.

The term “alkyl,” as used herein, describes groups that are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The terms “aryl” or “Ar,” as used herein, alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

### EXAMPLE

Implementation of the present disclosure is provided by way of the following example. The example serves to illustrate the technology without being limiting in nature.

### Example 1: Dose Dependent Viability Assays

To measure the effects of TC-E 5008 on cell viability, breast cancer cells were seeded in 96-well plates (4×103 cells/well) in RPMI medium containing 10% fetal bovine serum. After an overnight incubation, cells were treated with varying concentrations of TC-E 5008 or other mutant IDH1 inhibitors for 72 hours. Viability was measured using Cell Titer-Glo Luminescent Cell Viability Assay (Promega) in 96-well, flat, clear-bottom, opaque-wall micro plates according to manufacturer's protocol. IC50 was less than 2 mM in ER+ breast cancer cell lines and greater than 10 mM in ER− breast cancer cell lines, as shown in the Figures.

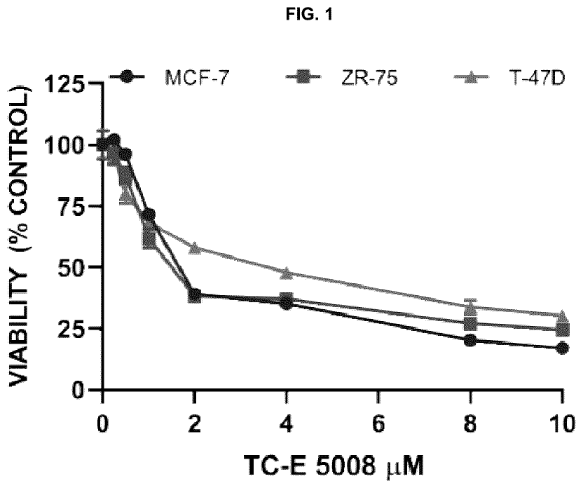

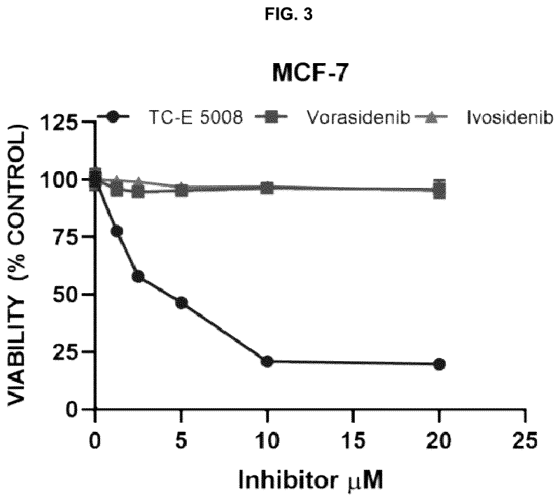

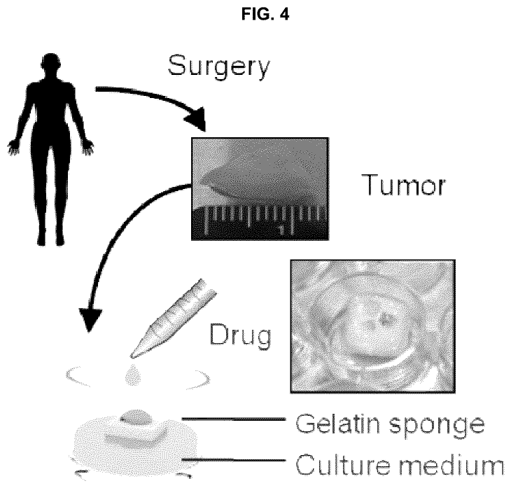

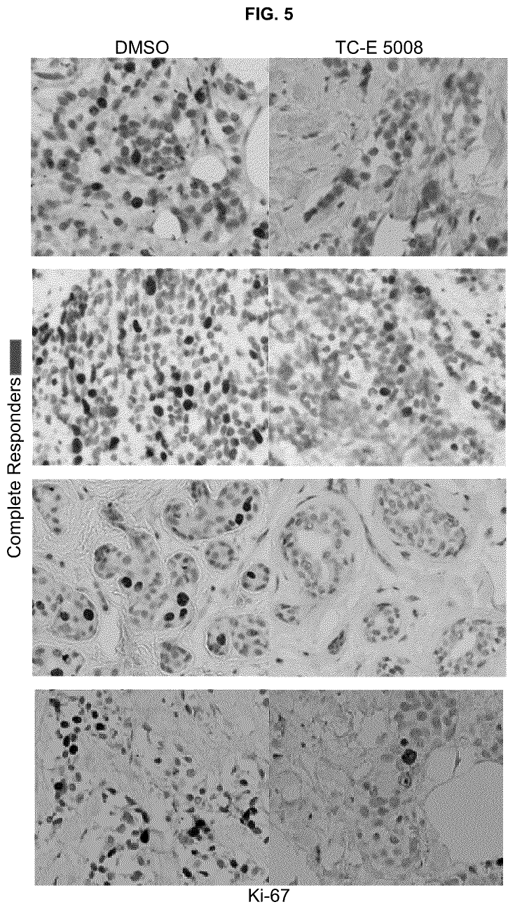

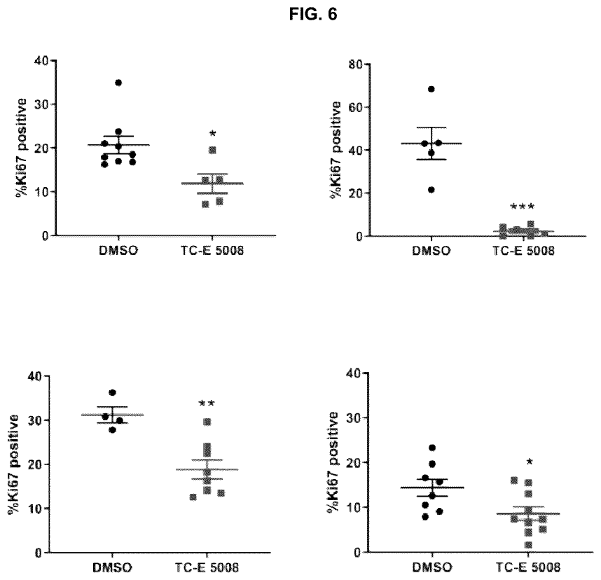

The Cell Titer-Glo assay showed anti-proliferative activity of TC-E 5008 compound on ER positive breast cancer cells (as shown in FIG. 1) and showed that TC-E 5008 and mutant IDH1 inhibitors on MCF-7 cells (as shown in FIG. 3). FIG. 4 is a schematic representation of patient-derived explant (PDE) model. The effect of TC-E 5008 (10 μM) on Ki-67 expression in ER+ tumor explants is shown in FIGS. 5-7. PDE data shown for complete responders in FIG. 6 shows the mean+SEM of individual Ki-67 positive cell counts in multiple fields of view with (*p<0.05,**p<0.01,***p<0.005 relative to DMSO using Student's unpaired T-test).

### Example 2: Dose Dependent Viability Assays

Excised tissue samples were processed and cultured ex vivo. De-identified tumors were obtained from the University of Texas Southwest Tissue Repository after institutional review board approval. Briefly, tumor samples were incubated on gelatin sponges for 24 hours in culture medium containing 10% fetal bovine serum (hereafter “FBS”), followed by treatment with either vehicle, 10 μM TC-E 5008 for 72 hours. Representative tissues were fixed in 10% formalin at 4° C. overnight and subsequently processed into paraffin blocks. The sections were then processed for immunohistochemical analysis. There was a significant decrease in proliferative indices in ER+ breast cancer tumors for primary tumor explants cultured ex vivo with the drug for 72 hour.

### Example 3: Compounds of Formula (II) Effect on ER+ Breast Cancer Cells

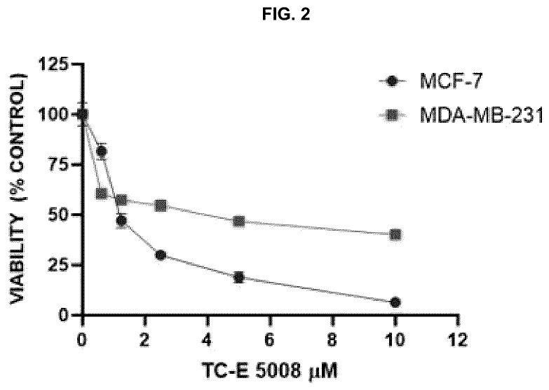

A compound having a structure in accordance with Formula II (sometimes referred to herein as “TC-E 5008”) was selected for validation with an integrated confidence score of 1362.6. Three commonly used ER-positive breast cancer cell lines MCF-7, ZR-75 and T-47D were used for in vitro validation. The selected compound (TC-E 5008) blocked proliferation of all three ER-positive breast cancer cell lines (see FIG. 1), but showed less activity in a triple negative breast cancer cell line (see FIG. 2). Two similar inhibitors that were not predicted to have activity were also evaluated, and indeed they did not block proliferation in MCF-7 cells (see FIG. 3).

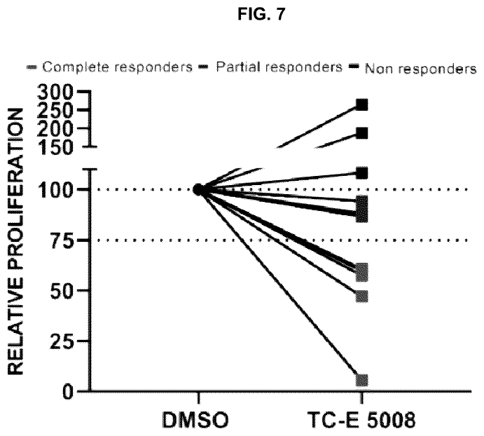

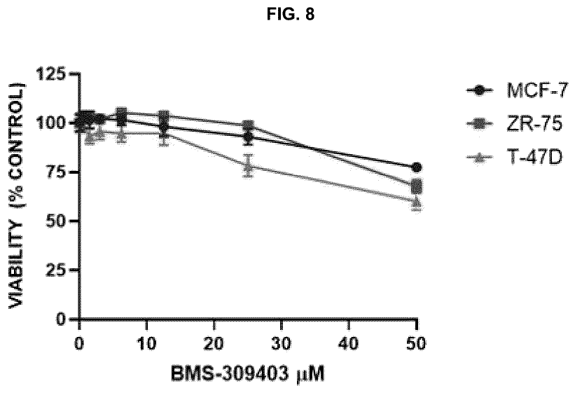

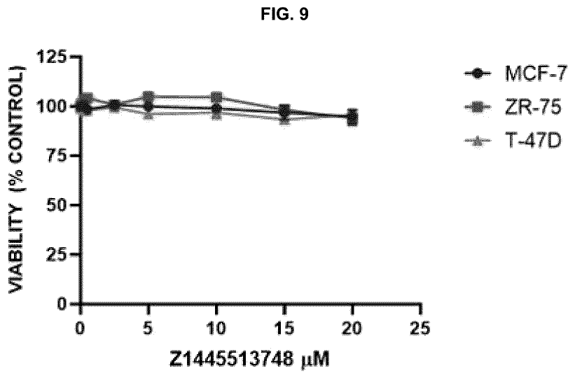

In order to further validate TC-E 5008, an ex vivo culture model of primary breast tumors was used—which allows for the validation of drugs on breast tumors in their native tissue architecture. Specifically, surgically resected breast tissues were sliced into small pieces and grown ex vivo for short term on a gelatin sponge in the absence or presence of desired compound (see FIG. 4). In order to test TC-E 5008 in a variety of ER-positive tumors, estrogen receptor positivity was the only inclusion criteria used for the breast tumor samples. Incubation of TC-E 5008 with ER-positive breast tumors samples decreased their proliferation (Ki-67 staining) in 7/10 patients with 4/10 showing a significant reduction ranging from 40-95% (Complete responders) (see FIGS. 5 and 6) and 3/10 showing a reduction in Ki-67 staining between 0-25% (Partial responders). Three tumor tissues showed an increase in proliferation (Non-responders) when compared to untreated controls (see FIG. 7). We did not observe morphological changes in the patient tissues treated with TC-E 5008 when compared to controls. Apart from TC-E 5008, two other predicted compounds were tested, which did not show activity in our three ER-positive cell lines (see FIGS. 8 and 9).

In the limited cohort evaluated in this Example, a 40% response rate suggests that TC-E 5008 has the potential to impact the growth of human breast tumors expressing ER. A range of anti-proliferative activity of TC-E 5008 is anticipated since patient stratification was kept to a minimum.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A compound of formula (I), an acid thereof, or a salt thereof: 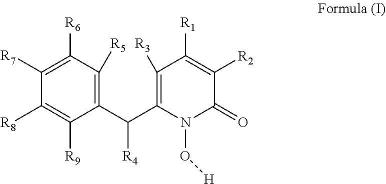
wherein
R₁ is a methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl, and
R₂, R₃, R₄, R₅, R₆, R₇, R₈, and R₉, are independent selected from hydrogen, methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl.

2. The compound of claim 1, wherein R₁ is a methyl.

3. The compound of claim 1, wherein R₂, R₃, R₄, R₅, R₆, R₇, R₈, and R₉, are independently selected from hydrogen or methyl.

4. The compound of claim 1, wherein at least one of R₂, R₃, R₄, R₅, R₆, R₇, R₈, and R₉ is hydrogen.

5. The compound of claim 4, wherein R₂, R₃, R₄, R₅, R₆, R₇, R₈, and R₉ are hydrogen.

6. The compound of claim 1, wherein the compound of Formula (I) has a structure according to Formula (II):

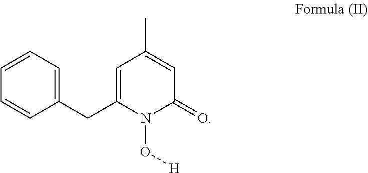

7. A composition comprising:
an amount a compound of formula (I), an acid thereof, or a salt thereof: 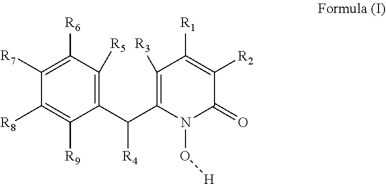
wherein
R₁ is a methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl, and
R₂, R₃, R₄, R₅, R₆, R₇, R₈, and R₉, are independent selected from hydrogen, methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl.

8. The composition of claim 7 further comprising:
at least one excipient.

9. The composition of claim 7, wherein the amount the compound of formula (I) is therapeutically effective.

10. A method for treating breast cancer in a subject in need thereof comprising:
administering an effect amount of a compound of formula (I), an acid thereof, or a salt thereof, wherein the compound of formula (I) has the structure: 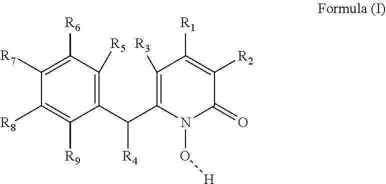
wherein
R₁ is a methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl, and
R₂, R₃, R₄, R₅, R₆, R₇, R₈, and R₉, are independent selected from hydrogen, methyl, C₂ to C₁₀ unsubstituted aryl, C₂ to C₁₀ substituted aryl, C₁ to C₁₀ unsubstituted alkyl, C₁ to C₁₀ substituted alkyl, C₃-C₁₀ unsubstituted cycloalkyl, or C₃-C₁₀ substituted cycloalkyl.

11. The method of claim 10, wherein R₁ is a methyl.

12. The method of claim 10, wherein R₂, R₃, R₄, R₅, R₆, R₇, R₈, and R₉, are independently selected from hydrogen or methyl.

13. The method of claim 10, wherein at least one of R₂, R₃, R₄, R₅, R₆, R₇, R₈, and R₉ is hydrogen.

14. The method of claim 13, wherein R₂, R₃, R₄, R₅, R₆, R₇, R₈, and R₉ are hydrogen.

15. The method of claim 10, wherein the compound of Formula (I) has a structure according to Formula (II):

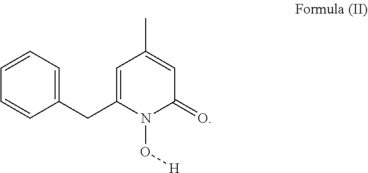

16. The method of claim 10, wherein the breast cancer is estrogen receptor positive (ER+) breast cancer.
